(19) United States
(12) Patent Application Publication
(10) Pub. No.: US 2024/0115169 A1
(43) Pub. Date: Apr. 11, 2024

(54) AUXILIARY IMPLANTATION DEVICE FOR BIOSENSOR

(71) Applicants: Shaoda ZHANG, Guangdong (CN); Wei ZHENG, Guangdong (CN); Xingyu ZHENG, Guangdong (CN)

(72) Inventors: Shaoda ZHANG, Guangdong (CN); Wei ZHENG, Guangdong (CN); Xingyu ZHENG, Guangdong (CN)

(21) Appl. No.: 18/206,087

(22) Filed: Jun. 6, 2023

(30) Foreign Application Priority Data
Oct. 11, 2022 (CN) 202211239570.7

(51) Int. Cl. A61B 5/145; A61B 17/34

(52) U.S. Cl. CPC A61B 5/14503; A61B 5/14532; A61B 17/3468; A61B 2560/063

(57) ABSTRACT

The present invention discloses an auxiliary implantation device for a biosensor. The auxiliary implantation device for the biosensor includes an emitting tube assembly, a battery assembly, and an emitter assembly. The emitting tube assembly is configured to drive a slider to emit a sensor to a human body through an elastic force and pull back the slider through the elastic force to enable the slider to be rapidly separated from the sensor. The battery assembly is configured to secure the sensor and supply power to the whole device. The emitter assembly is assembled with the battery assembly and is electrically connected with the sensor. According to the present invention, the rapid implantation of the sensor and the automatic needle withdrawal may be realized, without an external force applied by a user. Therefore, the risk of needle withdrawal failure caused by misoperation is avoided.

### BACKGROUND OF THE INVENTION

The present invention relates to the field of equipment for implanting biosensors, in particular to an auxiliary implantation device for a biosensor.

Diabetes mellitus is a syndrome of metabolic disorders of carbohydrate, fat and protein caused by relative or absolute insulin deficiency and different degrees of insulin resistance, with persistent hyperglycemia being its biochemical feature. As the population ages, eating disorders, physical inactivity, and continuous increase of obese populations, the incidence of diabetes will be higher and higher in both developed and developing countries.

Therefore, there is a need for a method capable of continuously monitoring blood glucose. Patients can know their blood glucose status at any time and take timely measures to control the disease most effectively, prevent complications, and achieve a higher quality of life. In order to obtain real-time data, a method for measuring tissue fluid is a continuous monitoring means available in reality. When sensors are implanted into blood vessels as a daily device carried by patients, high risks such as infection or blood loss will not be introduced, necessary data can be effectively provided, and therefore, the method for measuring tissue fluid has become an important direction of clinical monitoring development

The Chinese invention patent with the authorization number of CN103750818B discloses a subcutaneous implantable biosensor, which is implanted into the subcutaneous tissue for detection. The existing implantable biosensor is very small, and when the implantable biosensor is implanted into the skin, the implantable biosensor is wrapped by a hard needle tube and transmitted into the subcutaneous tissue, and then the hard needle tube is withdrawn to leave the implantable biosensor in the subcutaneous tissue.

When a hard needle tube and an implantable biosensor are implanted in the existing product, the used implanter is low in implanting speed and inaccurate in implanting position. The implanting and withdrawing time is long during implanting, so that a user has a tingling sensation, and the pain of the user is increased. The low implanting speed will also cause damage to subcutaneous tissue and adversely affect the detection effect of the implantable biosensor.

### BRIEF SUMMARY OF THE INVENTION

In order to overcome the defects that the current auxiliary implanter has use risks, for example, an ejection mechanism performs ejection too early due to misoperation in the use process; a hard needle is easy to prick a user; when the hard needle stays in the body for too long, the user has obvious tingling feeling, and the pain feeling and foreign body sensation of the user are increased; and accidental injury is easy to be caused during use. The prevent invention provides an auxiliary implantation device for a biosensor.

The technical solution of the present invention is as follows:
an auxiliary implantation device for a biosensor includes:
an emitting tube assembly configured to drive a slider to emit a sensor to a human body through an elastic force and pull back the slider through the elastic force to enable the slider to be rapidly separated from the sensor;
a battery assembly configured to secure the sensor and supply power to the whole device; and
an emitter assembly assembled with the battery assembly, electrically connected with the sensor, and transmitting a monitoring signal of the sensor to wireless terminal equipment.

According to the present invention adopting the above invention, the emitting tube assembly includes an ejecting tube, an emitting spring, a needle stand, a needle withdrawal spring, and a slider.

Both the needle stand and the slider are positioned inside the ejecting tube and slide up and down along the ejecting tube, the slider is clamped with the needle stand and is capable of rotating relative to the needle stand, the slider is also clamped with the sensor, and a vertically extending end of the sensor is sleeved with a guide needle at the bottom of the needle stand.

The emitting spring is positioned between the ejecting tube and the needle stand, the upper end of the emitting spring is abutted against the top of the ejecting tube, the lower end of the emitting spring is abutted against the slider, and the emitting spring is configured to drive the needle stand and the slider to eject along the ejecting tube.

The needle withdrawal spring is positioned inside the needle stand, the top of the needle withdrawal spring is abutted against the top end of the needle stand, the bottom of the needle withdrawal spring is abutted against the slider, and the needle withdrawal spring is configured to drive the needle stand to rebound.

Furthermore, the ejecting tube includes an ejecting tube body and an ejecting fastener positioned on the ejecting tube body, and when the slider is not in an ejecting state, the ejecting fastener is clamped with the slider.

Furthermore, the ejecting fastener includes a pressing part and a hooking part that are positioned at two opposite ends. The pressing part is suspended relative to the ejecting tube body. The hooking part is clamped with the slider.

Furthermore, a slider outer clamping block protrudes from the outer side of the slider, and the slider outer clamping block is clamped with the hooking part, so that when the slider is not in an ejecting state, the slider is clamped with the ejecting fastener.

Furthermore, the outer side of the ejecting tube body is sleeved with a limiting ring, the limiting ring includes a limiting ring body as well as a closing limiting protrusion and an unlocking area which are positioned on the limiting ring body, and the inner surface of the pressing part is abutted against the closing limiting protrusion, or the pressing part is suspended at the periphery of the unlocking area.

Furthermore, a protruding strip for clamping and securing the ejecting tube protrudes from a lower end of the ejecting tube body, an arc-shaped groove running through from top to bottom is formed in a battery upper cover of the battery assembly, a protruding strip for clamping and securing the upper cover protrudes from the arc-shaped groove, and the protruding strip for clamping and securing the upper cover is clamped with the protruding strip for clamping and securing the ejecting tube.

Furthermore, a guide post extending downwards is disposed at the top of the ejecting tube body, a guide slot running through from top to bottom is formed at the top of the needle stand, and the guide post extends into the guide slot, so that the needle stand slides up and down along the guide post.

Furthermore, the needle stand includes a needle stand body configured to drive a sensor to be implanted and realize needle withdrawal, and a guide needle positioned at the lower end of the needle stand and configured to guide the sensor. The lower end of the needle stand body is provided with an opening. A center post extending downwards is disposed at the top end inside the needle stand body. A needle stand inner protrusion is disposed at the bottom of the center post. The slider is internally provided with a slider inner clamping block corresponding to the needle stand inner protrusion. When the needle stand and the slider are in an ejection state, the slider inner clamping block is buckled with the needle stand inner protrusion. When the needle stand is in a rebound state, the slider inner clamping block is dislocated and separated from the needle stand inner protrusion.

Furthermore, an assembly groove through which the sensor passes is formed at the bottom of the slider, and an assembly clamping strip through which the sensor is clamped protrudes from the side wall of the assembly groove.

According to the above solution, the present invention has the beneficial effects that through the application of a plurality of buckle structures, emitting springs and needle withdrawal springs, the rapid implantation of the sensor and the automatic needle withdrawal may be realized, and the risk of needle withdrawal failure caused by misoperation is avoided; meanwhile, the convenience of single-hand operation is realized through a miniaturized design; and the springs and the buckles in an implanter are automatically unlocked and released in the process of puncturing and recovering the guide needle, so that a user does not need to apply external force, and the use is more convenient.

### BRIEF DESCRIPTION OF THE DRAWINGS

FIG. 1 is a schematic diagram of a connection between an emitting tube assembly and a battery assembly according to the present invention;

FIG. 2 is a structural exploded view of an emitting tube assembly and a battery assembly according to the present invention;

FIG. 3 is a schematic diagram of an emitting tube assembly before emitting a sensor according to the present invention;

FIG. 4 is a schematic diagram of an emitting tube assembly after emitting a sensor according to the present invention;

FIG. 5 is a schematic diagram of an emitting tube assembly after emitting a sensor from another perspective according to the present invention;

FIG. 6 is a cross-sectional view of an emitting tube assembly according to the present invention;

FIG. 7 is a cross-sectional view of an emitting tube assembly from another perspective according to the present invention;

FIG. 8 is an exploded view of an emitting tube assembly according to the present invention;

FIG. 9 is a structural schematic diagram of an ejecting tube according to the present invention;

FIG. 10 is a schematic diagram of an ejecting tube from another perspective according to the present invention;

FIG. 11 is a structural schematic diagram of a needle stand according to the present invention;

FIG. 12 is a schematic diagram of a needle stand from another perspective according to the present invention;

FIG. 13 is a structural schematic diagram of a slider according to the present invention;

FIG. 14 is a schematic diagram of a slider from another perspective according to the present invention;

FIG. 15 is a structural schematic diagram of a sensor and a probe base according to the present invention;

FIG. 16 is a structural schematic diagram of a limiting ring according to the present invention;

FIG. 17 is a schematic diagram of a connection between a battery assembly and an emitter assembly according to the present invention;

FIG. 18 is a schematic diagram of a connection between a battery assembly and an emitter assembly from another perspective according to the present invention;

FIG. 19 is an exploded view of a battery assembly and an emitter assembly according to the present invention;

FIG. 20 is an exploded view of a battery assembly according to the present invention;

FIG. 21 is a schematic diagram of a battery assembly after disassembling from another perspective according to the present invention;

FIG. 22 is a structural schematic diagram of a battery upper cover according to the present invention;

FIG. 23 is an exploded view of an emitter assembly according to the present invention;

FIG. 24 is a schematic diagram of an emitter assembly after disassembling from another perspective according to the present invention.

In the drawing, the respective reference numerals are:
100. emitting tube assembly;
110, ejecting tube; 111, ejecting tube body; 112, ejecting fastener; 1121, pressing part; 1122, hooking part; 113, slideway; 114, guide slope; 115, needle withdrawal limiting groove; 116, protruding strip for clamping and securing ejecting tube; 117, groove ring; 118, inner chute; 119, guide post;
120, emitting spring;
130. needle stand; 131. needle stand body; 1311. guide slot; 1312. center post; 1313. guide chute; 1314. needle stand inner protrusion; 132. guide needle; 1321. tip; 1322. guide groove;
140. needle withdrawal spring;
150. slider; 151. slider body; 152. slider outer clamping block; 153. slider inner clamping block; 154. assembly groove; 155. assembly clamping strip;
160. sensor;
170, probe base; 171, probe base body; 172, first sensor through hole; 173, sensor clamping and securing part; 174, assembly part; 1741, assembly clamping point;
180. limiting ring; 181. limit ring body; 182. closing limiting protrusion; 183. opening limiting protrusion; 184. unlocking area;
200. battery assembly;
210, battery upper cover; 211, upper cover hooking edge; 212, upper cover accommodating groove; 213, second sensor through hole; 214, first electrode plate clamping point; 215, first electrode plate through hole; 216, second electrode plate through hole; 217, ejecting tube clamping strip; 2171, clamping strip protruding point; 218, protruding strip for clamping and securing upper cover;
220, battery lower cover; 221, third sensor through hole; 222, first electrode plate supporting table; 223, second electrode plate supporting table; 224, second electrode plate clamping point; 230, battery;
240, first electrode plate;
250, second electrode plate;
300, emitter assembly;
310, emitter upper cover;
320, emitter base; 321, base hooking groove; 322, base accommodating groove;
330, PCBA (Printed Circuit Board Assembly); 331, metal elastic sheet; 332, elastic sheet contact;
340. sealing ring.

### DETAILED DESCRIPTION OF THE INVENTION

The present invention will be further described below with reference to the accompanying drawings and embodiments.

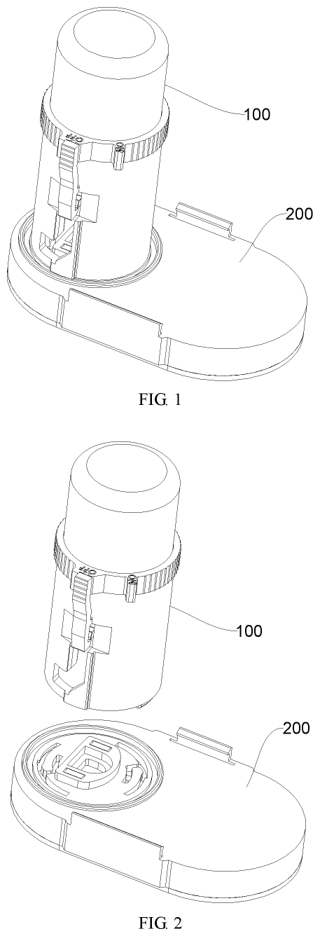

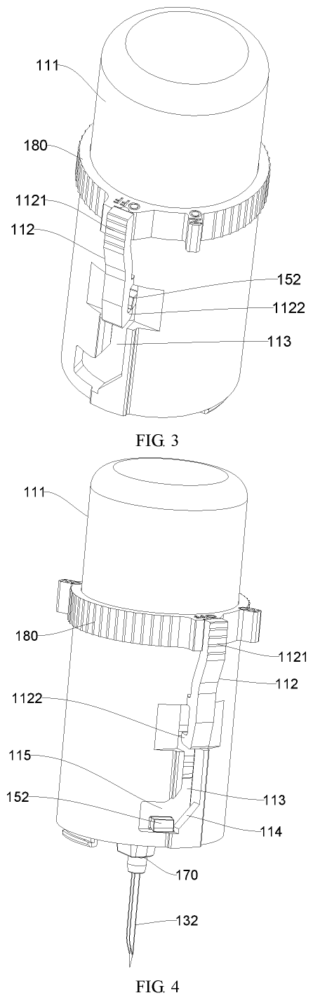

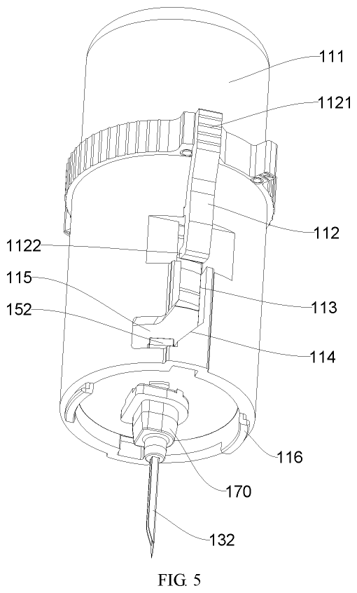

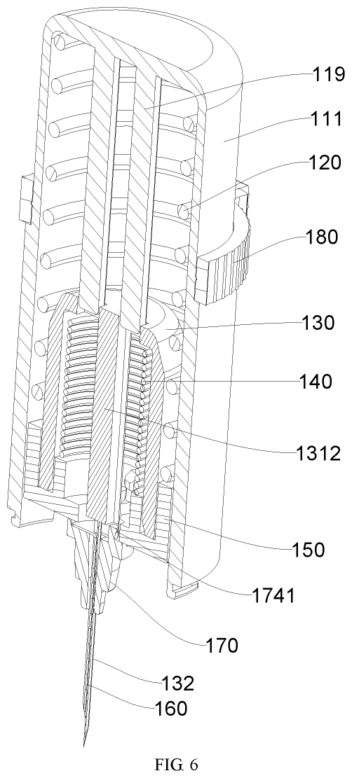

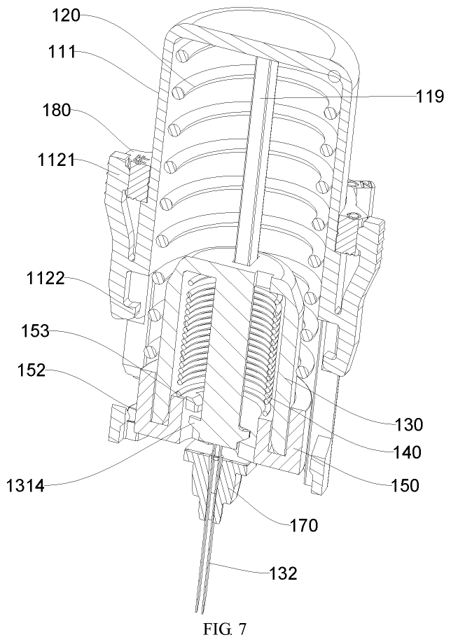

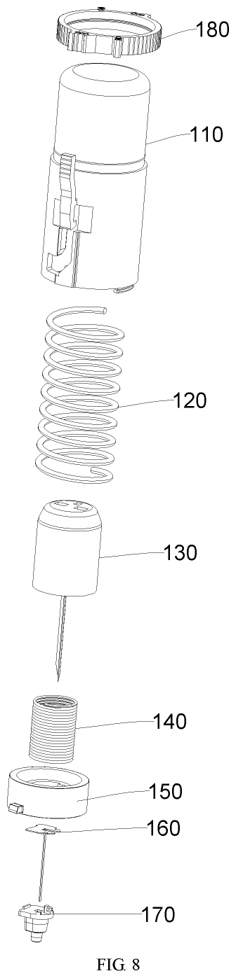

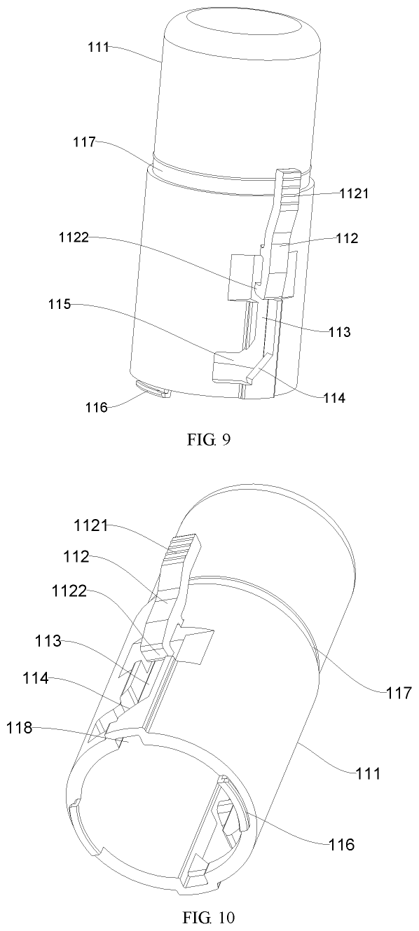

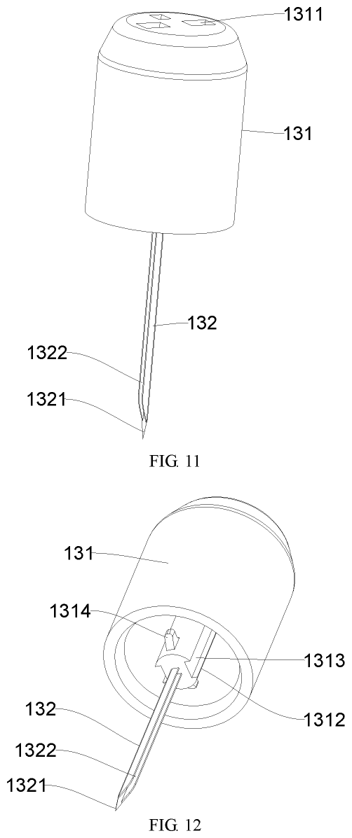

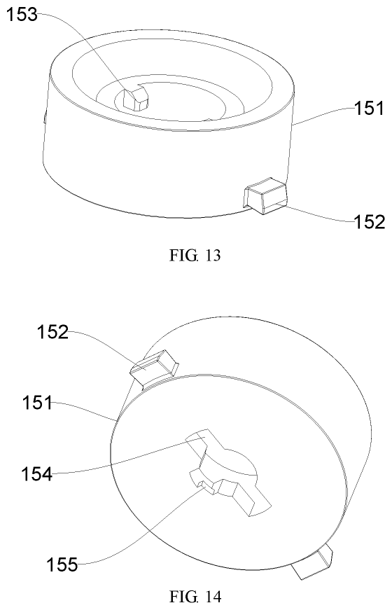

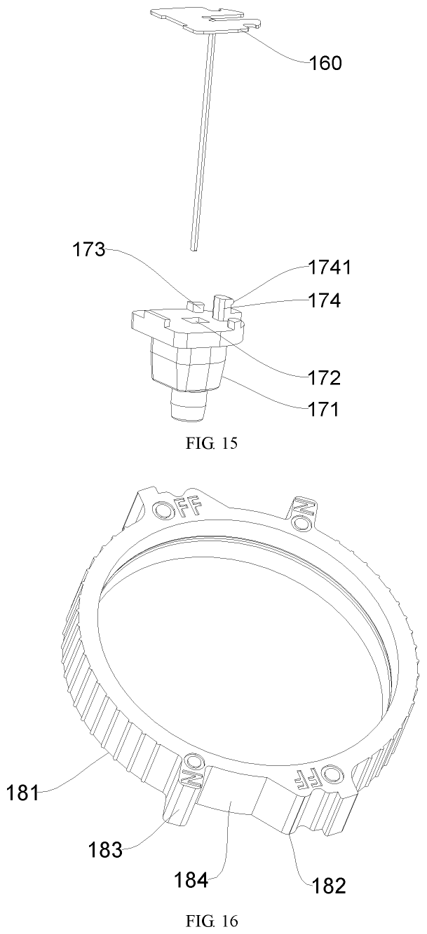

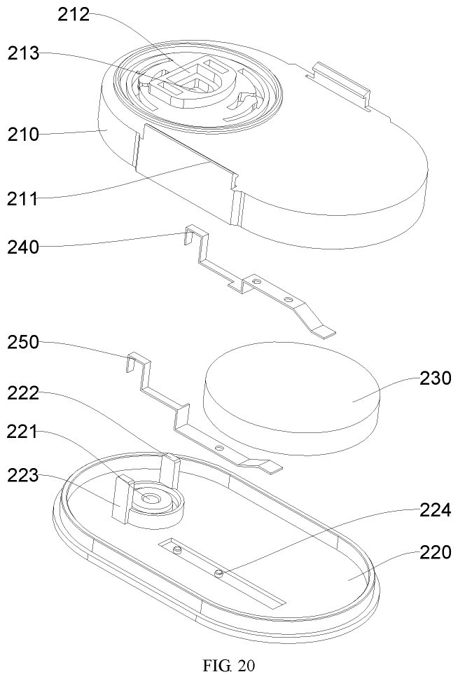

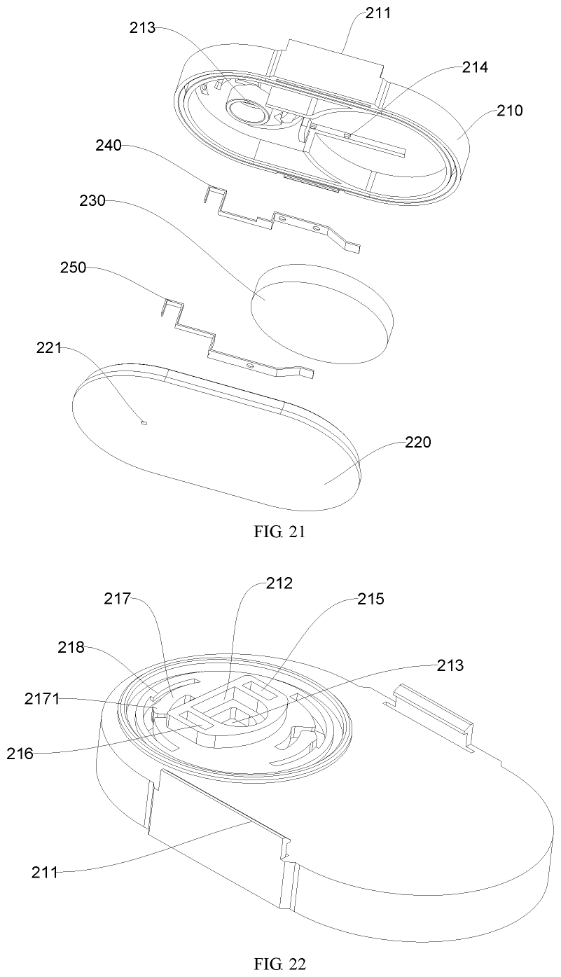

As shown in FIG. 1 to FIG. 24, according to the present invention, a subcutaneous tissue fluid of a user may be monitored, such as blood sugar. In order to avoid the use risk of an existing auxiliary implantation device for a sensor, an auxiliary implantation device for a biosensor is provided. The auxiliary implantation device for the biosensor includes an emitting tube assembly 100, a battery assembly 200, and an emitter assembly 300. The emitting tube assembly 100 is configured to drive a slider 150 to emit a sensor to a human body through an elastic force and pull back the slider 150 through the elastic force to enable the slider 150 to be rapidly separated from the sensor. The battery assembly 200 is configured to secure the sensor and supply power to the whole device. The emitter assembly 300 is assembled with the battery assembly 200, is electrically connected with the sensor, and transmits a monitoring signal of the sensor to wireless terminal equipment.

During use, the emitting tube assembly 100 is first assembled on the battery assembly 200, and then the battery assembly 200 is positioned on the epidermis of the human body. The sensor is implanted under the skin of the human body through the emitting tube assembly 100, and then the emitting tube assembly 100 and the battery assembly 200 are detached, and the emitter assembly 300 is assembled on the battery assembly 200. In the subsequent process, the emitter assembly 300 transmits blood sugar data monitored by the sensor to wireless terminal equipment (such as a mobile phone, which is wirelessly connected to the emitter assembly 300 through Bluetooth, WIFI, etc.) for monitoring.

I. Emitting Tube Assembly

As shown in FIG. 1 to FIG. 16, the emitting tube assembly 100 includes an ejecting tube 110, an emitting spring 120, a needle stand 130, a needle withdrawal spring 140, and a slider 150. The ejecting tube 110 is configured to provide a basis for the auxiliary implantation process of the sensor 160 and the needle withdrawal process. The emitting spring 120 is configured to provide power for the auxiliary implantation process of the sensor 160 and realize rapid implantation. The needle stand 130 is configured to position the sensor 160 and drive the sensor 160 to realize implantation. The needle withdrawal spring 140 is configured to provide power for the needle withdrawal process of the needle stand 130. The slider 150 is configured to drive the needle stand 130 and the sensor 160 to be implanted under the action of the emitting spring 120 and provide a supporting force for the needle withdrawal process of the needle stand 130.

1. Ejecting Tube

The ejecting tube 110 includes an ejecting tube body 111 and an ejecting fastener 112 positioned on the ejecting tube body 111. When the slider 150 is not in an ejecting state, the ejecting fastener 112 is clamped with the slider 150. Specifically, the ejecting fastener 112 includes a pressing part 1121 and a hooking part 1122 that are positioned at two opposite ends. When the auxiliary implantation is required, the pressing part 1121 is suspended relative to the ejecting tube body 111, the hooking part is clamped with the slider 150, and after the pressing part 1121 is pressed down, the hooking part is separated from the slider 150 at the clamped position.

The outer side of the ejecting tube body 111 is sleeved with a limiting ring 180. The limiting ring 180 includes a limiting ring body 181 as well as a closing limiting protrusion 182, an opening limiting protrusion 183 and an unlocking area 184 which are positioned on the limiting ring body 181. The unlocking area 184 is positioned between the opening limiting protrusion 183 and the closing limiting protrusion 182. Both the closing limiting protrusion 182 and the opening limiting protrusion 183 protrude from the surface of the limiting ring body 181, so that the unlocking area 184 is recessed relative to the closing limiting protrusion 182 and the opening limiting protrusion 183. When the ejecting fastener 112 is in a locking state, the inner surface of the pressing part 1121 is abutted against the closing limiting protrusion 182. When the ejecting fastener 112 is in an unlocking state, the pressing part 1121 is suspended at the periphery of the unlocking area 184. According to the present invention, the ejecting fastener 112 may be locked or unlocked through the limiting ring 180, thereby ensuring that the situation of false triggering does not occur during transportation. Meanwhile, the misoperation in the use process may be prevented, thereby avoiding the secondary damage.

Preferably, in order to limit the ejecting fastener 112, a groove ring 117 protrudes from the periphery of the ejecting tube body 111 in the present invention, a concave ring corresponding to the groove ring 117 is disposed on the inner side of the limiting ring body 181, and the limiting ring body 181 is clamped with the periphery of the ejecting tuber body 111 through the matching of the concave ring and the groove ring 117.

In order to realize the ejection and needle withdrawal of the slider 150, the needle stand 130 and other structures, the ejecting tube body 111 is provided with a track for guiding the movement of the slider 150, and the slider 150 is provided with a corresponding sliding clamping part. Specifically, the sliding clamping part is a slider outer clamping block 152 protruding from the outer side of the slider 150, and the track includes a slideway 113 extending up and down along the ejecting tube body 111, and a needle withdrawal limiting groove 115 formed in the lower end of the slideway 113 and extending transversely, where the needle withdrawal limiting groove 115 is connected to the slideway 113 through an inclined guide slope 114. When the slider 150 is not in an ejecting state, the slider outer clamping block 152 is clamped with the hooking part 1122, so that the slider 150 is clamped with the ejecting fastener 112. When the slider 150 is ejected, the slider outer clamping block 152 slides downwards along the slideway 113, and is clamped with the needle withdrawal limiting groove 115 when sliding downwards to the bottom end, so that the slider 150 does not rebound when the needle stand 130 rebounds after needle withdrawal.

Preferably, in order to enable the slider 150 to be smoothly assembled in the ejecting tube body 111, an inner sliding groove 118 is formed the inner side surface of the ejecting tube body 111, and the inner chute 118 is aligned with the extending direction of the slideway 113, so that the slider outer clamping block 152 slides into the slideway 113 from the inner sliding groove 118.

A guide post 119 extending downwards is disposed at the top of the ejecting tube body 111, and is configured to guide the implantation ejecting and needle withdrawal rebounding of the needle stand 130, so as to prevent the needle stand 130 from skewing in the process of ejection and rebound to affect the implantation quality of the sensor 160 and the safety of the human body.

A protruding strip 116 for clamping and securing the ejecting tube protrudes from a lower end of the ejecting tube body 111. The ejecting tube body 111 is clamped with the battery assembly 200 through the protruding strip 116 for clamping and securing the ejecting tube and is separated after the sensor 160 is implanted.

2. Emitting Spring

The emitting spring 120 is positioned between the ejecting tube 110 and the needle stand 130, the upper end of the emitting spring 120 is abutted against the top of the ejecting tube 110, the lower end of the emitting spring 120 is abutted against the slider 150, and the emitting spring 120 is configured to drive the needle stand 130 and the slider 150 to eject along the ejecting tube 110.

In the implementation process, the emitting spring 120 is in a compressed state, so that the elastic potential energy of the emitting spring 120 is converted into the kinetic energy of the slider 150 and the needle stand 130, and then the sensor 160 may be rapidly implanted under the skin of a human body. Because the elastic distribution of the emitting spring 120 is more uniform, the slider 150 and the needle stand 130 may be more stable in the process of ejection.

3. Needle Stand

The needle stand 130 is positioned inside the ejecting tube 110 and slides up and down along the ejecting tube 110, and includes a needle stand body 131 configured to drive the sensor 160 to be implanted and realize the needle withdrawal, and a guide needle 132 positioned at the lower end of the needle stand 130 and configured to guide the sensor 160, where the lower end of the needle stand body 131 is open, a center post 1312 extending downwards is disposed at the top end inside the needle stand body 131, and the guide needle 132 is positioned at the bottom end of the center post 1312. A body part of the guide needle 132 is provided with a guide groove 1322, so that the section of the whole guide needle 132 is U-shaped. A vertically extending end of the sensor 160 is sleeved with the guide groove 1322, so that the sensor 160 may be implanted under the skin of a human body along with the guide groove 1322. A tip 1321 is disposed at the lower end of the guide needle 132, and is formed in such a way that the bottom wall of the guide groove 1322 extends forwards, and the tip 1321 may pierce the epidermis of a human body to facilitate the rapid implantation.

In cooperation with the guide post 119 inside the ejecting tube body 111, in the present invention, a guide slot 1311 running through from top to bottom is formed at the top of the needle stand 130, and the guide post 119 extends into the guide slot 1311, so that the needle stand 130 slides up and down along the guide post 119. Preferably, a vertically extending guide chute 1313 is formed inside the needle stand 130, and is opposite to the guide slot 1311. The guide post 119 may slide up and down along the guide chute 1313. The guide post 119 is always in a state of being attached to the guide chute 1313 in the sliding process of the needle stand 130. By matching the insertion position of the guide post 119 and the guide slot 1311, the needle stand 130 can be ensured to slide in the same direction, thereby further increasing the stability smoothness of the needle seat 130.

In order to realize the synchronous ejection of the needle stand 130 and the slider 150, a needle stand inner protrusion 1314 is disposed at the bottom of the center post 1312 of the present invention. The needle stand inner protrusion 1314 is configured in such a way that when the needle stand 130 and the slider 150 are in an assembly and ejection state, the needle stand inner protrusion 1314 is clamped with the slider 150, and when the needle stand 130 is in a rebound state, the needle stand inner protrusion 1314 is separated from the slider 150.

4. Needle Withdrawal Spring

The needle withdrawal spring 140 is positioned inside the needle stand 130, the top of the needle withdrawal spring 140 is abutted against the top end of the needle stand 130, the bottom of the needle withdrawal spring 140 is abutted against the slider 150, and the needle withdrawal spring 140 is in a compressed state, and is configured to drive the needle stand 130 and the sensor 160 to rebound.

In the implementation process, as the slider 150 is limited by the needle withdrawal limiting groove 115, the bottom position of the needle withdrawal spring 140 is not changed, and the top end thereof provides a rebound force for the inner side top end of the needle stand 130. After the needle stand 130 is separated from the slider 150, rebound is realized under the action of the needle withdrawal spring 140.

5. Slider

The slider 150 is positioned inside the ejecting tube 110 and slides up and down along the ejecting tube 110. The slider 150 is clamped with the needle stand 130 and can rotate relative to the needle stand 130, so as to realize the linkage and rebound of the slider 150 and the needle stand 130. The slider 150 of the present invention includes a slider body 151, the slider body 151 includes a hollow cylindrical outer side wall and an inner side wall, a cavity between the outer side wall and the inner side wall is used for being matched with the bottom of the needle stand 130, and a slider outer clamping block 152 is positioned on the outer side of the outer side wall of the slider body 151.

The slider 150 is internally provided with a slider inner clamping block 153 corresponding to a needle stand inner protrusion 1314 in the needle stand 130. When the needle stand 130 and the slider 150 are in an assembly and ejection state, the slider inner clamping block 153 is buckled with the needle stand inner protrusion 1314 of the needle stand 130. When the needle stand is in a rebound state, the slider inner clamping block 153 is dislocated and separated from the needle stand inner protrusion 1314. In the specific implementation process, when the slider 150 and the needle stand 130 are assembled, the slider 150 slides into the slideway 113 under the interaction of the slider outer clamping block 152 and the inner chute 118 of the ejecting tube body 111, and at this time, the slider inner clamping block 153 is opposite to and clamped with the needle stand inner protrusion 1314. As the needle stand 130 does not rotate in the circumferential direction, the slider 150 will rotate in the circumferential direction under the action of the guide slope 114 and slide into the needle withdrawal limiting groove 115 after ejection, and at this time, the slider inner clamping block 153 is dislocated and separated from the needle stand inner protrusion 1314.

In addition, the needle stand 130 can only guide the implantation of the sensor 160, and the slider 150 is clamped with the sensor 160, so that the sensor 160 is driven to be implanted by the slider 150, thereby providing power for the implantation process of the sensor 160. An assembly groove 154 for the sensor 160 to pass through is formed at the bottom of the slider 150 of the present invention, and an assembly clamping strip 155 through which the sensor 160 is clamped protrudes from the side wall of the assembly groove 154.

The sensor 160 made of a flexible material is clamped with the slider 150 through the probe base 170, and after the implantation is completed, the probe base 170 is assembled in the battery assembly 200 and the emitter assembly 300, so that the positioning effect of the sensor 160 may be realized through the probe base 170. The probe base 170 includes a probe base body 171 and a first sensor through hole 172 which vertically penetrates the probe base body 171 and allows the sensor 160 to pass through. The vertically extending end of the sensor 160 passes through the first sensor through hole 172. Preferably, a plurality of sensor clamping and securing parts 173 are further disposed above the probe base body 171, and the top of the sensor 160 is limited within the range of the sensor clamping and securing parts 173, so that the probe base 170 may provide a stable limiting basis for the sensor 160, thereby preventing the sensor 160 from shaking to affect the implantation effect.

In addition, an assembly part 174 protrudes from the upper end of the probe base body 171, and the assembly part 174 is provided with an assembly clamping point 1741. When the slider 150 and the sensor 160 are in an ejection state, the probe base 170 is clamped with the assembly clamping strip 155 through the assembly clamping point 1741. When the probe base 170 is in a rebound state, the assembly clamping point 1741 is dislocated and separated from the assembly clamping strip 155.

II. Battery Assembly

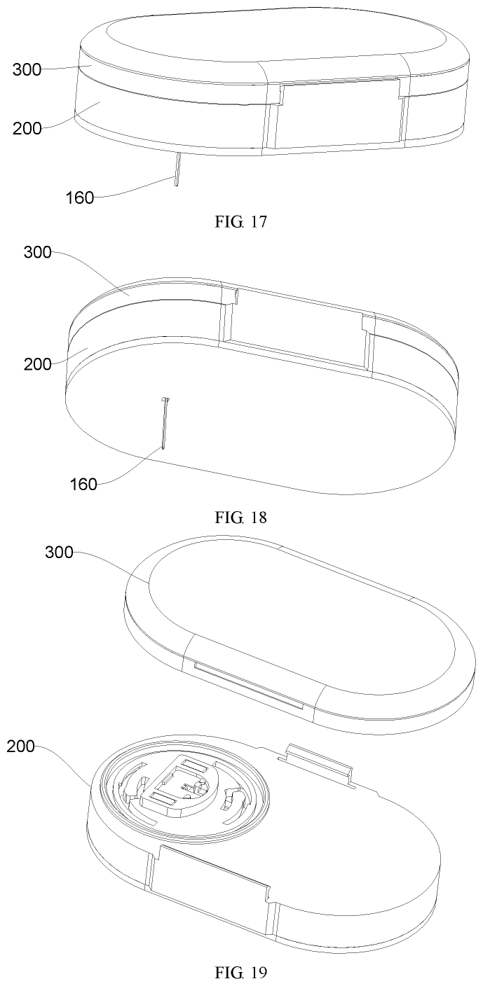

As shown in FIG. 17 to FIG. 22, the battery assembly 200 provides a positioning basis for the sensor 160, the probe base 170, and provides electrical power for the entire auxiliary implantation device for the biosensor. The battery assembly 200 includes a battery upper cover 210, a battery lower cover 220, a battery 230, a first electrode plate 240, and a second electrode plate 250, where one end of the first electrode plate 240 is in contact with the battery 230, the other end of the first electrode plate 240 passes through the battery upper cover 210 and is electrically connected with the emitter assembly 300, one end of the second electrode plate 250 is in contact with the battery 230, and the other end of the second electrode plate 250 is electrically connected with the emitter assembly 300 through the battery upper cover 210. In the embodiment, the first electrode sheet 240 is a positive electrode plate, and the second electrode plate 250 is a negative electrode plate.

In order to facilitate the assembly of the first electrode plate 240 and the second electrode plate 250, and to realize the positioning of the first electrode plate 240 and the second electrode plate 250, a first electrode plate clamping groove configured to position the first electrode plate 240 is formed on the top of the inner side of the battery upper cover 210, a first electrode plate clamping point 214 configured to fasten the first electrode plate 240 is disposed in the first electrode plate clamping groove, a second electrode plate clamping groove configured to position the second electrode plate 250 is formed on the inner bottom side of the battery lower cover 220, and a second electrode plate clamping point 224 configured to fasten the second electrode plate 250 is disposed in the second electrode plate clamping groove.

In addition, the battery upper cover 210 is provided with a first electrode plate through hole 215 configured to accommodate the first electrode plate 240 to pass through and a second electrode plate through hole 216 configured to accommodate the second electrode plate 250 to pass through. The battery lower cover 220 is provided with a first electrode plate supporting table 222 configured to support the first electrode plate 240, and a second electrode plate supporting table 223 configured to support the second electrode plate 250, where the position of the first electrode plate supporting table 222 corresponds to that of the first electrode plate through hole 215, and the position of the second electrode supporting table 223 corresponds to that of the second electrode plate through hole 216. After the assembly, the end of the first electrode plate 240 is supported on the first electrode plate supporting table 222 and exposed from the first electrode plate through hole 215, and the end of the second electrode plate 250 is supported on the second electrode plate supporting table 223 and exposed from the second electrode plate through hole 216.

The battery assembly 200 may also position the sensor 160 and the probe base 170. Specifically, an upper cover accommodating groove 212 sunken downwards is formed in the upper surface of the battery upper cover 210, and is configured to accommodate and secure the probe base 170; and the battery upper cover 210 is provided with a second sensor through hole 213 running through from top to bottom, the battery lower cover 220 is provided with a third sensor through hole 221 running through from top to bottom, and a vertically extending end of the sensor 160 is implanted into the skin of a human body after penetrating through the second sensor through hole 213 and the third sensor through holes 221.

In the process of implanting the sensor 160, the battery assembly 200 provides a positioning effect, and therefore, the battery assembly 200 also needs to be clamped with the emitting tube assembly 100. Specifically, the battery upper cover 210 of the battery assembly 200 is provided with an arc-shaped groove running through from top to bottom, and a protruding strip 218 for clamping and securing the upper cover protrudes from the arc-shaped groove, so that when the battery assembly 200 and the ejecting tube 110 are in an assembly state, the protruding strip 116 for clamping and securing the ejecting tube passes through the arc-shaped groove and is clamped with the protruding strip 218 for clamping and securing the upper cover; and after the implantation of the sensor 160 is completed, the ejecting tube 110 is screwed, so that the protruding strip 116 for clamping and securing the ejecting tube is separated from the protruding strip 218 for clamping and securing the upper cover. Preferably, an elastic ejecting tube clamping strip 217 is disposed on the inner side of the arc-shaped groove, a clamping strip protruding point 2171 protrudes from the outer side end of the ejecting tube clamping strip 217, and the clamping strip protruding point 2171 is configured to limit the protruding strip 116 for clamping and securing the ejecting tube, so that the ejecting tube 110 and the battery upper cover 210 are more tightly limited to avoid separation.

The battery upper cover 210 is further provided with an upper cover hooking edge 211 configured to be clamped with the emitter assembly 300, an emitter base 320 of the emitter assembly 300 is provided with a base hooking groove 321 corresponding to the position of the upper cover hooking edge 211, and the upper cover hooking edge 211 is connected with the base hooking groove 321, so that the battery assembly 200 is clamped with the emitter assembly 300.

The battery assembly 200 further includes a skin-friendly adhesive tape. One side of the skin-friendly adhesive tape is connected with the lower surface of the battery lower cover 220, and the other side is configured to be in contact with the epidermis of a human body, so as to achieve the purpose that the battery assembly 200 is tightly attached to the surface of a human body.

III. Emitter Assembly

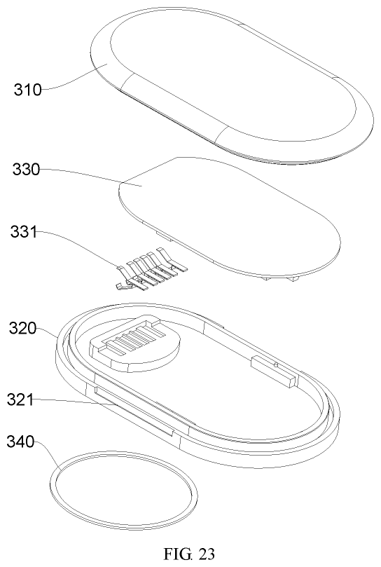

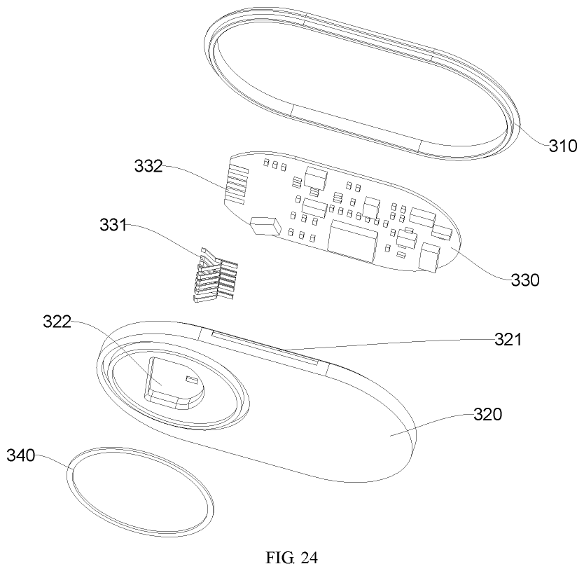

As shown in FIG. 17 to FIG. 19, FIG. 23, and FIG. 24, the emitter assembly 300 includes an emitter upper cover 310, an emitter base 320, and a PCBA 330 positioned between the emitter upper cover 310 and the emitter base 320. The PCBA 330 is provided with an elastic sheet contact 332 configured to be in contact and connected with a metal elastic sheet 331. The metal elastic sheet 331 is connected with the emitter base 320 by injection molding. The electrode elastic sheet in the metal elastic sheet 331 is in contact with the first electrode plate 240 and the second electrode plate 250 in the battery assembly 200. In addition, the PCBA 330 may communicate with the wireless terminal equipment of a user (for example, Bluetooth communication), and is configured to transmit the detection result of the sensor 160 to the wireless terminal equipment for data analysis and display. The functions of the PCBA 330, such as receiving data from the sensor 160 and transmitting data to the wireless terminal equipment, are well known in the art, and improvements are not made in the present invention, and therefore, a specific implementation circuit of the PCBA 330 will not described in detail.

A base accommodating groove 322 sunken upwards, which corresponds to the position of the upper cover accommodating groove 212 of the battery assembly 200, is formed in the lower side of the emitter base 320, and the base accommodating groove 322 is configured to accommodate and secure the probe base 170, so that the probe base 170 is positioned in a space surrounded by the upper cover accommodating groove 212 and the base accommodating groove 322.

Preferably, a sealing ring 340 is disposed on the lower side of the emitter base 320, and the emitter base 320 is hermetically connected with the battery assembly 200 through the sealing ring 340. Specifically, a sealing groove is formed at the bottom of the emitter base 320, and the sealing ring 340 is embedded in the sealing groove; and a protruding ring protrudes from the upper surface of the battery upper cover 210, and extends into the sealing groove to be abutted against the sealing ring 340, so as to achieve a good sealing effect and avoid affecting the detection sensitivity of the sensor 160 in the long-term monitoring process.

The assembly process of the present invention is as follows:

1. Assembly of the battery assembly 200
pasting one side of a skin-friendly adhesive tape to the bottom of a battery lower cover 220;
securing a second electrode plate 250 in a second electrode plate clamping groove of the battery lower cover 220;
installing a button battery, so that a negative electrode of the battery is abutted against the second electrode plate 250;
securing a first electrode plate 240 in a first electrode plate clamping groove of a battery upper cover 210; and
closing the battery upper cover 210, so that a positive electrode of the battery is abutted against the first electrode plate 240.

2. Assembly of the emitter assembly 300
fastening a sealing ring 340 in a sealing groove of an emitter base 320; and
putting a PCBA 330 in the emitter base 320, and putting an emitter upper cover 310 on the emitter base 320.

3. Assembly of the emitting tube assembly 100
assembling a limiting ring 180 at a groove ring 117 from the top of the ejecting tube body 111, and adjusting the direction of the limiting ring 180 until the closing limiting protrusion 182 is buckled with the pressing part 1121;
putting an emitting spring 120 in the emitting tube body, and putting the needle withdrawal spring 140 in the needle stand body 131, so as to prevent the needle withdrawal spring 140 from being in contact with the guide needle 132 when installing the needle withdrawal spring 140, and to prevent the guide needle 132 from being damaged to affect the assembly and implantation effect;
covering the bottom of the needle stand 130 with a slider 150, and pressing the slider 150, so that the slider inner clamping block 153 is buckled with a needle stand inner protrusion 1314, and then compressing the needle withdrawal spring 140;
putting the needle stand 130, together with the slider 150, inside an ejecting tube body 111 and an ejecting spring 120, pressing the slider 150, so that the slider outer clamping block 152 slides into a slideway 113 along an inner chute 118, and continuously pressing the slider 150 and compressing the emitting spring 120 until clamping the slider outer clamping block 152 with the hooking part 1122 of the ejecting fastener 112;
assembling the sensor 160 on the probe base 170, so that the vertically extending end of the sensor 160 passes through a first sensor through hole 172; and
passing the guide needle 132 through the first sensor through hole 172, so that the vertically extending end of the sensor 160 is put in the guide groove 1322, and then clamping an assembly clamping point 1741 of the probe base 170 with the assembly clamping strip 155 of the slider 150.

3. Assembly of the Battery Assembly 200 and the Emitting Tube Assembly 100
putting the battery assembly 200 on the lower side of the emitting tube assembly 100, so that the guide needle 132, together with the vertically extending end of the sensor 160, passes through a second sensor through hole 213; and
pressing or rotating the battery assembly 200, so that a protruding strip 116 for clamping and securing the ejecting tube passes through an arc-shaped groove and is clamped with a protruding strip 218 for clamping and securing the upper cover, thereby completing the assembly of the battery assembly 200 and the emitting tube assembly 100.

The present invention further provides an implementation method of the auxiliary implementation method of the biosensor, The implementation method specifically includes:
S1, Unlocking

In the initial state, each assembly is positioned in a sterile package.

The sterile package is opened, a protective layer of a skin-friendly adhesive tape is torn off, and the battery assembly 200 is aligned to a part of a human body where the sensor 160 is required to be implanted; and a limiting ring 180 is rotated, so that a pressing part 1121 of an ejecting fastener 112 is adjusted to an unlocking area 184.
S2, Implanting
S21, The pressing part 1121 of the ejecting fastener 112 is pressed, so that a hooking part 1122 of the ejecting fastener 112 is unlocked from the slider outer clamping block 152;
S22, The slider 150, together with the needle stand 130 and the sensor 160, is rapidly pressed down under an elastic pushing force of the emitting spring 120, and a vertically extending end of the sensor 160 is punctured into the subcutaneous tissue under the guiding action of the guide needle 132;
S23, When the slider 150 slides down to the lowest end, the slider outer clamping block 152 moves along a track of a slideway 113, a guide slope 114 and a needle withdrawal limiting groove 115, and rotates at the guide slope 114 and the needle withdrawal limiting groove 115, and meanwhile, a slider inner clamping block 153 is separated from and unlocked from a needle stand inner protrusion 1314, and an assembly clamping strip 155 of the slider 150 is separated and unlocked from an assembly clamping point 1741 of a probe base 170; and
S24, The needle stand 130 rebounds rapidly under an elastic pushing force of a needle withdrawal spring 140, and meanwhile, the slider 150 does not rebound under an elastic force of the emitting spring 120 and a limiting effect of the needle withdrawal limiting groove 115.

The duration of the implantation process is extremely short, so that rapid implantation and needle withdrawal can be realized, and the pain of a user can be effectively alleviated. Through the positioning effects of the ejecting tube 110 and the battery assembly 200, the implantation position is accurate without deviation, and secondary damage to a human body is not easily caused.
S3, Supplying power
S31, An ejecting tube body 111 is rotated, so that a protruding strip 116 for clamping and securing the ejecting tube is separated from a protruding strip 218 for clamping and securing the upper cover of the battery assembly 200, and the ejecting tube body 111 is pulled out; and
S32, An emitter assembly 300 is placed on the battery assembly 200, so that a base hooking groove 321 of an emitter base 320 is connected with an upper cover hooking edge 211 of a battery upper cover 210, and meanwhile, the top of the probe base 170 is sealed between the emitter assembly 300 and the battery assembly 200, and the battery assembly 200 supplies power to the PCBA 330 inside the emitter assembly 300.
S4, Using

Wireless terminal equipment is connected to the PCBA 330 (for example, the PCBA 330 is connected through Bluetooth) and an emitter is activated to start working, and data collected by the sensor 160 and the PCBA330 are transmitted to the wireless terminal equipment.

According to the present invention, a mode that an internal spring and a buckle are automatically unlocked and released is adopted to realize rapid implantation and automatic needle withdrawal, so that a user has no obvious tingling feeling; and the user is not required to apply an external force, children and the elderly may use the auxiliary implantation device by themselves under the guidance of a guardian, the whole process is rapid and accurate, deviation is not easy to occur, and secondary damage to the user cannot be caused. In addition, a safety device and a securing buckle are added to ensure that the situation of false triggering does not occur in the hands of consumers during transportation.

It should be understood that modifications and variations may be made in light of the above description by those skilled in the art, and all such modifications and variations should fall within the scope of protection of the appended claims.

The patent of the present invention has been described by way of example above with reference to the accompanying drawings. Obviously, the implementation of the patent of the present invention is not limited by the above methods. As long as various improvements are made by using the method concept and technical solution of the patent of the present invention, or the concept and technical solution of the patent of the present invention are directly applied to other occasions without improvement, all fall within the scope of protection of the present invention.

What is claimed is:

1. An auxiliary implantation device for a biosensor, comprising:
an emitting tube assembly configured to drive a slider to emit a sensor to a human body through an elastic force and pull back the slider through the elastic force to enable the slider to be rapidly separated from the sensor;
a battery assembly configured to secure the sensor and supply power to the whole device; and
an emitter assembly assembled with the battery assembly, electrically connected with the sensor, and transmitting a monitoring signal of the sensor to wireless terminal equipment.

2. The auxiliary implantation device for a biosensor according to claim 1, wherein the emitting tube assembly comprises an ejecting tube, an emitting spring, a needle stand, a needle withdrawal spring, and a slider,
wherein both the needle stand and the slider are positioned inside the ejecting tube and slide up and down along the ejecting tube, the slider is clamped with the needle stand and is capable of rotating relative to the needle stand, the slider is also clamped with the sensor, and a vertically extending end of the sensor is sleeved with a guide needle at a bottom of the needle stand;
the emitting spring is positioned between the ejecting tube and the needle stand, an upper end of the emitting spring is abutted against a top of the ejecting tube, a lower end of the emitting spring is abutted against the slider, and the emitting spring is configured to drive the needle stand and the slider to eject along the ejecting tube; and
the needle withdrawal spring is positioned inside the needle stand, a top of the needle withdrawal spring is abutted against a top end of the needle stand, a bottom of the needle withdrawal spring is abutted against the slider, and the needle withdrawal spring is configured to drive the needle stand to rebound.

3. The auxiliary implantation device for a biosensor according to claim 2, wherein the ejecting tube comprises an ejecting tube body and an ejecting fastener positioned on the ejecting tube body, and when the slider is not in an ejecting state, the ejecting fastener is clamped with the slider.

4. The auxiliary implantation device for a biosensor according to claim 3, wherein the ejecting fastener comprises a pressing part and a hooking part that are positioned at two opposite ends, wherein the pressing part is suspended relative to the ejecting tube body, and the hooking part is clamped with the slider.

5. The auxiliary implantation device for a biosensor according to claim 4, wherein a slider outer clamping block protrudes from the outer side of the slider, and the slider outer clamping block is clamped with the hooking part, so that when the slider is not in an ejecting state, the slider is clamped with the ejecting fastener.

6. The auxiliary implantation device for a biosensor according to claim 4, wherein the outer side of the ejecting tube body is sleeved with a limiting ring, the limiting ring comprises a limiting ring body as well as a closing limiting protrusion and an unlocking area which are positioned on the limiting ring body, and the inner surface of the pressing part is abutted against the closing limiting protrusion, or the pressing part is suspended at the periphery of the unlocking area.

7. The auxiliary implantation device for a biosensor according to claim 2, wherein a protruding strip for clamping and securing the ejecting tube protrudes from a lower end of the ejecting tube body, an arc-shaped groove running through from top to bottom is formed in a battery upper cover of the battery assembly, a protruding strip for clamping and securing the upper cover protrudes from the arc-shaped groove, and the protruding strip for clamping and securing the upper cover is clamped with the protruding strip for clamping and securing the ejecting tube.

8. The auxiliary implantation device for a biosensor according to claim 2, wherein a guide post extending downwards is disposed at a top of the ejecting tube body, a guide slot running through from top to bottom is formed at a top of the needle stand, and the guide post extends into the guide slot, so that the needle stand slides up and down along the guide post.

9. The auxiliary implantation device for a biosensor according to claim 2, wherein the needle stand comprises a needle stand body configured to drive the sensor to be implanted and realize the needle withdrawal, and a guide needle positioned at the lower end of the needle stand and configured to guide the sensor, wherein a lower end of the needle stand body is provided with an opening, a center post extending downwards is disposed at a top end inside the needle stand body, a needle stand inner protrusion is disposed at a bottom of the center post, a slider inner clamping block corresponding to the needle stand inner protrusion is disposed inside the slider, when the needle stand and the slider are in an ejecting state, the slider inner clamping block is buckled with the needle stand inner protrusion, and when the needle stand is in a rebounding state, the slider inner clamping block is dislocated and separated from the needle stand inner protrusion.

10. The auxiliary implantation device for a biosensor according to claim 2, wherein an assembly groove through which the sensor passes is formed at a bottom of the slider, and an assembly clamping strip through which the sensor is clamped protrudes from the side wall of the assembly groove.
